(19) United States
(12) Patent Application Publication
(10) Pub. No.: US 2023/0240781 A1
(43) Pub. Date: Aug. 3, 2023

(54) SYSTEM, METHOD, AND APPARATUS FOR HAND-CENTRIC CONTROLLER FOR THE ROBOTIC DIGITAL SURGICAL MICROSCOPE

(71) Applicant: Digital Surgery Systems, Inc., Goleta, CA (US)

(72) Inventor: George C. Polchin, Santa Barbara, CA (US)

(21) Appl. No.: 18/009,528

(22) PCT Filed: Jun. 11, 2021
(86) PCT No.: PCT/US21/37059

§ 371 Date: Dec. 9, 2022

Related U.S. Application Data
(60) Provisional application No. 63/038,254, filed on Jun. 12, 2020.

(51) Int. Cl. A61B 34/00; A61B 90/00; A61B 90/20; H04N 23/62; H04N 23/66; G06F 3/01

(52) U.S. Cl. CPC A61B 34/74; A61B 90/361; A61B 90/37; A61B 90/20; H04N 23/62; H04N 23/66; G06F 3/012; A61B 2090/367; A61B 2034/742; G06F 3/167

(57) ABSTRACT

The present disclosure relates generally a hand-centric controller that provides a user (e.g., surgeon) with the ability to control a number of microscope movement controls, non-movement microscope controls, image and color controls, media controls, and hyperspectral controls without having to reach beyond the space surrounding the surgical tool being used or the space surrounding the surgeon's hands. In some embodiments, the hand-centric controller is a limited button (e.g., one, two, three buttons) controller. In other embodiments, the hand-centric controller is an extended hand-centric controller. The hand-centric controller may be configured to provide microscope movement (e.g., x-y axis movement, lock-to-target movement, yaw movement, physical focus movement, and gross general movement), non-movement microscope control (e.g., zoom, focus, autofocus, and white light), image and color controls (e.g., next image and previous image modes), media controls (e.g., snapshot control, stop and start recording modes), and hyperspectral controls (e.g., DIR 800 on/off, light control, and playback, and DUV 400 on/off and light control).

### RELATED APPLICATIONS

This application claims priority to U.S. Provisional Application No. 63/038,254, filed Jun. 12, 2020, the entire disclosure of which is hereby incorporated herein by reference in its entirety.

### TECHNICAL FIELD

The present disclosure relates to robotic digital surgical microscopes, and more specifically to a hand-centric controller for a robotic digital surgical microscope.

### BACKGROUND

Surgeons are craftspeople who rely strongly on their hands to achieve their objectives during surgery. During surgical procedures for which a surgical microscope is used, control of the microscope can be achieved through various means including: the surgeon's hands; reliance on an assistant to operate some or all of the controls; a foot pedal/footswitch; voice control; head tracking; navigated instrument tracking; a mouth switch.

Existing surgeon microscope control systems can provide some control over microscope movement (i.e., x-y axis movement, lock-to-target movement, yaw movement, physical focus movement, and gross general movement), imaging aspects (i.e., zoom, focus, autofocus, and white light, next image and previous image modes), media (i.e., snapshot control, stop and start recording modes), and hyperspectral features (i.e., DIR 800 on/off, light control, and playback, and DUV 400 on/off and light control). However, a problem with surgeon hand-based systems is that the surgeon's hands are also used to perform the surgical procedure, operating all or most of the instruments used to treat the patient's pathology. This problem is more than one of convenience; Surgeons often spend up to 40% of a surgery making adjustments to the microscope. This is a significant amount of “surgical waste” and has a negative impact on patient outcomes, surgeon focus, and hospital return on investment.

Existing systems that rely on an assistant to operate the controls can provide microscope movement (i.e., x-y axis movement, lock-to-target movement, yaw movement, physical focus movement, and gross general movement), non-movement microscope control (i.e., zoom, focus, autofocus, and white light), image and color controls (i.e., next image and previous image modes), media controls (i.e., snapshot control, stop and start recording modes), and hyperspectral controls (i.e., DIR 800 on/off, light control, and playback, and DUV 400 on/off and light control) but in general surgeons prefer to move the microscopes themselves rather than have an assistant move it. Assistant command of non-movement (i.e., light, color, etc.) is generally accepted.

Existing foot pedal/footswitch systems can provide microscope movement (i.e., x-y axis movement, lock-to-target movement, yaw movement, physical focus movement, and gross general movement), non-movement microscope control (i.e., zoom, focus, autofocus, and white light), image and color controls (i.e., next image and previous image modes), media controls (i.e., snapshot control, stop and start recording modes), and hyperspectral controls (i.e., DIR 800 on/off, light control, and playback, and DUV 400 on/off and light control). However, existing foot pedal/footswitch systems suffer from cluttering of foot pedals, which are already in use in the operating room for existing devices, crowding the floor space around the surgical bed where the patient is located. Further, operating the foot pedal puts the surgeon “off balance” because the dexterous operation required by the pedal means the surgeon puts most of their body weight on the non-pedal-operating foot. Additionally, a large set of commands is difficult to implement and learn on the foot pedal, and it is not feasible to have line of sight to the control to mitigate this. Further, the joystick of most existing foot pedals uses binary switches and is not a true analog joystick meaning that only moves in one direction along the +/−X axis or the +/−Y axis or along the set of axes rotated clockwise 45 degrees from them (8 directions in all) are possible. Additionally, fine microscope movements (while still allowing more coarse adjustments) are also quite difficult to achieve with such a joystick design.

Existing head tracking systems can provide microscope movement (i.e., x-y axis movement, lock-to-target movement, and physical focus) and non-movement microscope control (i.e., focus in the z-movement). However, head tracking systems do not allow for an engage/disengage method so that user's head is only tracked when desired. Accordingly, unexpected motion of the robot could occur.

Existing voice control systems can provide non-movement microscope control (i.e., zoom, focus, autofocus, and white light), image and color controls (i.e., next image and previous image modes), media controls (i.e., snapshot control, stop and start recording modes), and hyperspectral controls (i.e., DIR 800 on/off, light control, and playback, and DUV 400 on/off and light control). However, existing voice control systems suffer from performance problems with respect to response time and compute load (often requiring network access) and are also prone to inaccurate voice recognition and high miss rates (e.g., “precision” and “recall”).

Existing navigated instrument tracking can provide microscope movement (i.e., x-y axis movement and slow gross general movement) and non-movement microscope control (i.e., focus). However, existing navigated instrument tracking requires bulky targets to be mounted on every instrument used, as well as unimpeded line-of-sight to the navigation camera.

Existing microscope mounted mouth-operated brake release systems can provide microscope movement (i.e., x-y axis movement, lock-to-target movement, yaw movement, physical focus movement, and gross general movement) and non-movement microscope control (focus). However, existing microscope mounted mouth-operated systems cannot do more than move the microscope around and requires surgeon head to remain in close proximity to microscope head for duration of use. Inserting the microscope in the mouth, in the operating room, is not a preferred ergonomic method of controlling a microscope. Thus, the use of this approach is shrinking.

To operate traditional control systems (as outlined above), alone or in combination, the surgeon must remove one or both of their hands from the surgical field, typically relinquishing control of the surgical instrument currently in use by that hand or hands. The relinquishment of one or both hands is disruptive to the progress of the procedure for the surgeon to operate in this manner, extending the amount of time the procedure takes, and interrupting the procedure workflow. Further, in some of the traditional control systems, the surgeon must relinquish control of an instrument (or of two instruments if both hands are employed and are both holding instruments), operate the microscope controls and then re-locate the instruments and pick them up again or re-accept them from the scrub nurse. Accordingly, after completion of using the control system, the surgeon needs to re-position the instrument(s) in the hand(s) to their preferred and/or necessary position in the hand.

### SUMMARY

The present disclosure relates generally a hand-centric controller that provides a user (e.g., surgeon) with the ability to control a number of microscope movement controls, non-movement microscope controls, image and color controls, media controls, and hyperspectral controls. In some embodiments, the hand-centric controller is a limited button (e.g., one, two, three buttons) controller. In other embodiments, the hand-centric controller is an extended hand-centric controller. The hand-centric controller may be configured to provide microscope movement (e.g., x-y axis movement, lock-to-target movement, yaw movement, physical focus movement, and gross general movement), non-movement microscope control (e.g., zoom, focus, autofocus, and white light), image and color controls (e.g., next image and previous image modes), media controls (e.g., snapshot control, stop and start recording modes), and hyperspectral controls (e.g., DIR 800 on/off, light control, and playback, and DUV 400 on/off and light control).

In an example, a hand-centric digital surgical microscope system includes a microscope camera showing, within its field of view, a surgical site; and a hand-centric controller for providing a user control of the microscope camera. The hand-centric controller may be located remotely from the microscope camera. For example, the microscope camera may be positioned to show the surgical site as its field of view, whereas the hand-centric controller may be closer to the bed rails of surgical site, affixed to a surgical tool used by the operator, or otherwise be in quick reach of the hands of the operator. The hand-centric controller may be attached or configured to be attachable to a surgical tool (e.g., a cannula, tweezers, forceps, electrocautery devices, etc.). Also or alternatively, the hand-centric controller may be attached or configured to be attachable to a hand of the user (e.g., a finger or a palm of the user).

The hand-centric controller may further include a plurality of buttons associated with a plurality of corresponding functions of the microscope camera. Activation of at least one of the plurality of buttons may cause, via digital communication, performance of a corresponding function of the microscope camera. The functions of the microscope camera may include, but are not limited to: a movement along an x, y, or z axis; a lock to target movement; a yaw movement; an increase or a decrease in lighting associated with the microscope camera; a zoom function; a focus function; a use of fluorescence; a use of color lighting; a recording function; a hyperspectral function; an image capture function; or a selection of an image mode. In some aspects, the plurality of buttons may include discrete buttons provided by a joystick. Each discrete button may correspond to (e.g., be activated by) a direction of a motion of the joystick. Activation of at least one of the plurality of discrete buttons may cause, via digital communication, a movement of the microscope camera in a direction corresponding to the direction of the motion of the joystick. The plurality of buttons may further include a joystick lock button, wherein activation of the joystick lock button restricts movement of the microscope camera.

The hand-centric controller may further include a fastening member to attach and detach the hand-centric controller with respect to the surgical tool. Examples of fastening members may include, for example, a zip tie, an adjustable strap or clip, an elastomeric strap having a channel to secure a surgical tool and a finger of the user in parallel, a combination of a front flap and a rear flap, or a sleeve. The hand-centric controller may further include a power module for providing power to the hand-centric controller via one or more of a rechargeable battery or a power supply. Furthermore, the hand-centric controller may include a programmable feedback module for communicating feedback from the microscope camera. The programmable feedback module can cause (e.g., on or via the hand-centric controller, and in response to feedback from the microscope camera) one or more of: a vibration, an acoustic feedback, a tactile feedback, a haptic feedback, or an indicator light (e.g., that is flashing or is turned “on” from a previously “off” state). The feedback from the microscope may include, for example, an inability to move further in a specific direction (e.g., in response to a user input requesting the microscope camera to move towards a restricted region), a low battery or power supply alert, a loss of or lack of connection with the hand-centric controller, or a status of being connected with the hand-centric controller.

In some aspects, the system may further include a head tracking device configured to detect head movements of the user and cause corresponding movements in the microscope camera in the direction of the head movements. Furthermore, the plurality of buttons may include an engage/disengage button. Activation of the engage/disengage button may cause the head tracking device to detect the head movements and cause the corresponding movements in the microscope camera. Deactivation of the engage/disengage button may prevent the head tracking device from detecting head movements and/or prevent the head tracking device from causing the corresponding movements in the microscope camera.

In another embodiment of the present disclosure, an example method of providing a user of a surgical tool with a hand-centric control of a microscope camera is disclosed. The method may include establishing, by a controller having one or more processors and located remotely from a microscope camera, a connection with the microscope camera. The controller may display, via an indicator light and based on an acknowledgment of the connection, an indication that the microscope camera is connected. A pairing module associated with the controller may pair the controller to the microscope camera, wherein the pairing prevents the microscope camera from being controlled by another controller during a time period associated with the pairing, and wherein the pairing prevents the controller from controlling another microscope camera during the time period associated with the pairing. The controller may receive, via a button of a plurality of buttons on the controller, a user input corresponding to a function of the microscope camera. The controller may determine, based on a mapping of the plurality of buttons to a plurality of functions of the microscope camera, the function corresponding to the user input. The controller may then send, to the microscope camera, a command to perform the function corresponding to the user input. In some aspects, before sending the command, the controller may determine whether it has permission to engage the microscope camera based on the user input. For example, an engage/disengage button may have been previously toggled to disengage to prevent certain user input (e.g., joystick movement, head tracking device movement) from causing the performance of one or more functions on the microscope camera. Alternatively, the engage/disengage button may have been toggled to engage (e.g., by a previous input by the user) to allow the controller to cause the microscope camera to perform one or more functions.

In another embodiment of the present disclosure, a system for a hand-centric controller for visualization is disclosed. The system comprises: a camera showing a surgical site; an actuated control system (e.g., that controls the camera's field of view); a hand-centric controller comprising at least one control element; and a fastening member. The hand-centric controller may provide a user control of the visualization and actuation systems via the at least one control element. Activation of the at least one control element can cause, via communication, execution of a predefined function. For example, the predefined function comprises one or more of: a movement along any of the six degrees of freedom of x, y, z, roll, pitch, or yaw; a movement on a hemisphere around the focal point; an increase or a decrease in lighting associated with the camera; a zoom function; a focus function; a use of fluorescence; a use of color lighting; a recording function; a hyperspectral function; an image capture function; an unlock button for actuated motion; or a selection of an image mode. The hand-centric controller may be located remotely from the camera. Furthermore, the hand-centric controller may be within a sterile field.

The fastening member may be used to attach the hand-centric controller, or render the hand-centric controller attachable, for example, to a surgical tool, a hand of the user, a railing of a bed, or a combination thereof. In some aspects, the fastening member may comprise a zip tie. The hand-centric controller may be attached by tightening the zip tie. The fastening member may further include a zip tie base, which can be used to easily attach and remove the hand-controller and/or zip tie from the zip tie base. In some aspects, the fastening member may include a front flap and a rear flap. The user may attach the surgical tool or tubing of the surgical tool to the hand-centric controller by gripping the front flap and the rear flap around the surgical tool. In some aspects, the fastening member may comprise a sleeve. The hand-centric controller may be attachable to the surgical tool or a tubing of the surgical tool via the sleeve enveloping the surgical tool or enveloping the tubing of the surgical tool. In some aspects, the fastening member may comprise a clip. The hand-centric controller may be attachable to the surgical tool or a tubing of the surgical tool via the clip hooking on to the surgical tool or the tubing of the surgical tool. In some aspects, the fastening member may comprise one or more of: an adjustable ring to enclose one or more of a finger of the user or a palm of the user, or an elastomeric strap around the hand of the user.

In some aspects, the at least one control element may include one or more of a joystick, a thumbstick, a directional button, or a track pad. The at least one control element may be arranged symmetrically so the hand-centric controller can be used identically by either hand.

In some aspects, the system may further include a programmable feedback module. The programmable feedback module can cause, via the hand-centric controller and in response to feedback from the camera, one or more of: a vibration, an acoustic feedback, a tactile feedback, a haptic feedback, or an indicator light.

In some aspects, the system may further include a pairing module configured to pair the hand-centric controller to the camera. The pairing can cause only one camera to be controlled by only one hand-centric controller.

In some aspects, the at least one control element may engage with one or more of the following control devices: a head tracking device configured to detect head movements, or a voice control device. The head tracking device may include 3D glasses configured to be worn by the user.

In some aspects, the at least one control element may generate a machine vision analysis of the camera image. The machine vision analysis can track a location of a tip of the surgical tool in the camera view.

In some aspects, the hand-centric controller may be disposable. In other aspects, the hand-centric controller reusable (e.g., sterilizable). Furthermore, the hand-centric controller can be sterile (e.g., inherently, or by being within a sterile field). Also or alternatively, the hand-centric controller can be draped (e.g., to provide sterility while in use).

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE FIGURES

FIG. 1 is a perspective view of a hand-centric controller for a left hand, according to an example embodiment.

FIG. 2 is a perspective view of a hand-centric controller for a right hand, according to another example embodiment.

FIG. 3 is a perspective view of the hand-centric controller for the left hand of FIG. 1 and the hand-centric controller for the right hand of FIG. 2

FIG. 4 is a perspective view of an expanded controls hand-centric controller for a left hand, according to another example embodiment.

FIG. 5 is a block diagram illustrating a hand-centric controller system, according to an example embodiment.

FIG. 6 is a table illustrating control components at various state and values for a hand-centric controller system, according to an example embodiment.

FIG. 7 is a table illustrating control components and corresponding states, values, and actions for microscopic movement of a hand-centric controller system, according to an example embodiment.

FIG. 8 is a table illustrating control components and corresponding states, values, and actions for non-microscopic movement microscope control of a hand-centric controller system, according to an example embodiment.

FIG. 9 is a table illustrating function, control components and corresponding states, values, and actions for image and color, media, and hyperspectral controls of a hand-centric controller system, according to an example embodiment.

FIG. 10 shows perspective views of a head tracking device mounted on 3D glasses with an integrated camera and light source module of a hand-centric controller system, according to an example embodiment.

FIG. 11 is a system diagram for head movement mapping of a hand-centric controller system, according to an example embodiment.

FIG. 12 shows perspective views of a hand-centric controller with a zip tie fastening mechanism, according to an example embodiment.

FIG. 13 shows perspective views of hand-centric controllers with a flex connector, according to example embodiments.

FIG. 14 shows perspective views of a hand-centric controller with an expandable channel, according to an example embodiment.

FIG. 15 shows perspective views of a hand-centric controller with a front flap and a rear flap, according to an example embodiment.

FIG. 16 shows perspective views of hand-centric controllers with sleeves, according to example embodiment.

FIG. 17 is a process flow diagram illustrating an example method of communication between the hand-centric controller and the microscope camera.

FIG. 18 is a block diagram illustrating a system for a hand-centric controller for visualization, according to an example embodiment.

### DETAILED DESCRIPTION OF EXAMPLE EMBODIMENTS

The present disclosure relates generally to a hand-centric controller that provides a user (e.g., surgeon) with the ability to control a number of microscope movement controls, non-movement microscope controls, image and color controls, media controls, and hyperspectral controls. In some embodiments, the hand-centric controller is a limited button (e.g., one, two, three buttons) controller. In other embodiments, the hand-centric controller is an extended hand-centric controller. The hand-centric controller may be configured to provide microscope movement (e.g., x-y axis movement, lock-to-target movement, yaw movement, physical focus movement, and gross general movement), non-movement microscope control (e.g., zoom, focus, autofocus, and white light), image and color controls (e.g., next image and previous image modes), media controls (e.g., snapshot control, stop and start recording modes), and hyperspectral controls (e.g., DIR 800 on/off, light control, and playback, and DUV 400 on/off and light control). The hand-centric control system of the present disclosure overcomes the problems with traditional control systems by alleviating the requirement by an operator (e.g., surgeon) to take time, focus, hands, and/or concentration away from surgery to operate the traditional control systems.

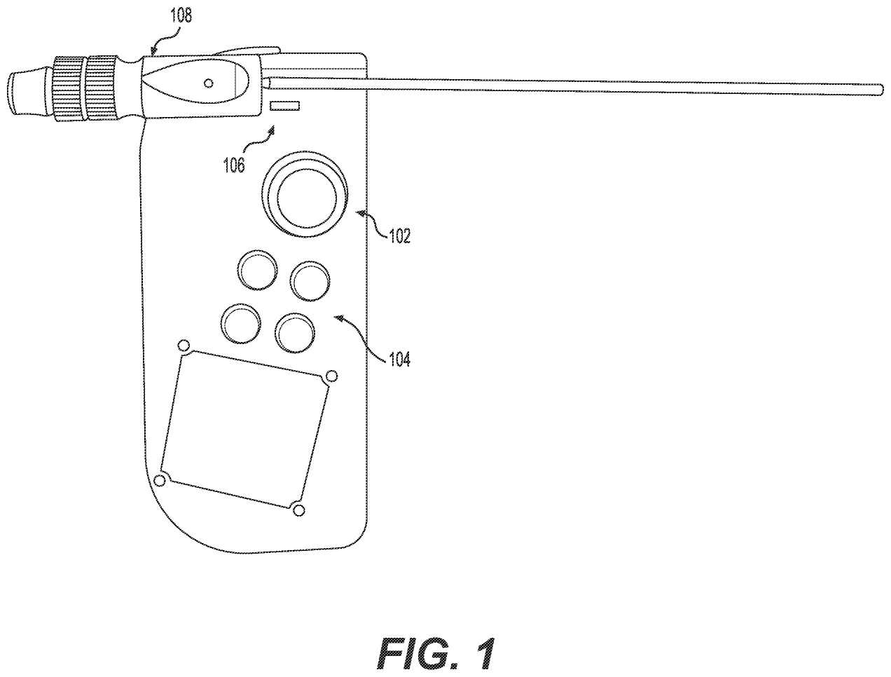

The hand-centric controller may be configured for use with a right hand, a left hand, or both hands. For example, FIG. 1 is a perspective view of a hand-centric controller for a left hand, according to an example embodiment. The hand-centric controller may include a base having an inner portion and an outer portion, a joystick 102 (e.g., a thumbstick, a movable cursor, etc.), a plurality of push buttons 104, and an indicator light 106. As shown in FIG. 1, joystick 102 and the plurality of push buttons 104 are arranged on one side, e.g., for the hand-centric controller to be suitable for use by a left hand of the operator. FIG. 1 further shows a surgical tool 108 for use by the operator (e.g., the surgeon), while the hand-centric controller is within reach (e.g., is reachable by the operator's fingers). In some embodiments, as will be discussed herein, the hand-centric controller may further include a fastening member. The fastening member may be used to secure, attach, or affix to a surgical tool (e.g., a cannula) or the hand or finger of the operator. In some embodiments, the hand-centric controller may comprise, or may be a part of, the surgical tool.

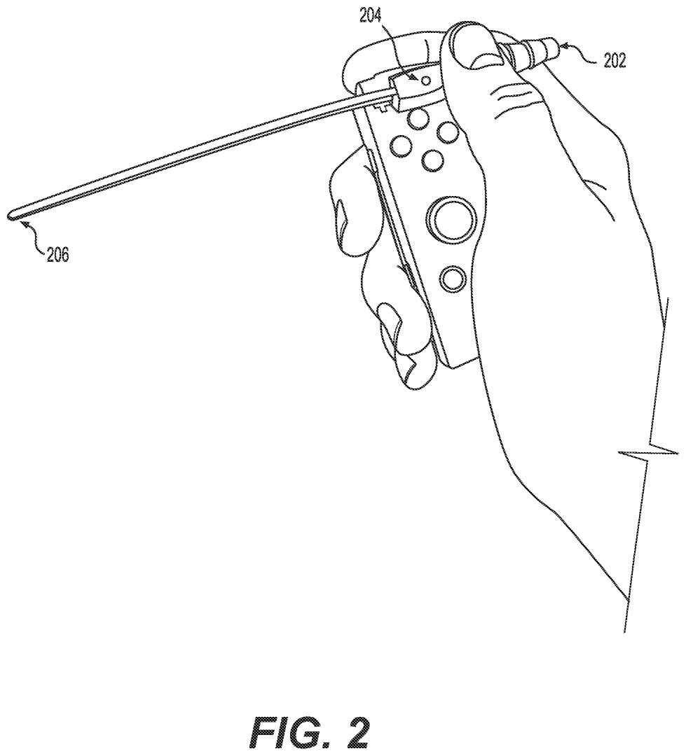

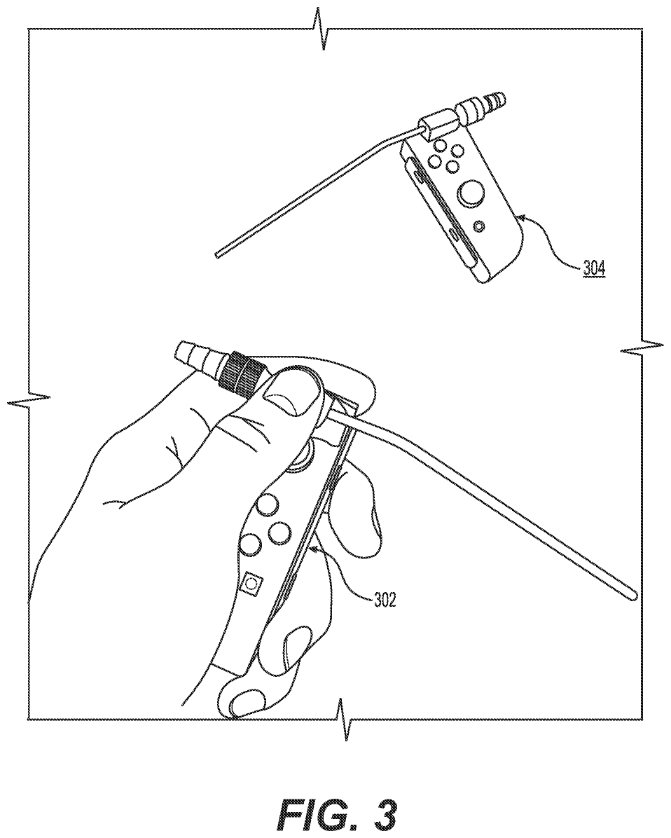

FIG. 2 is a perspective view of a hand-centric controller for a right hand, according to another example embodiment. For example, FIG. 2 shows how a right hand of an operator may be able to maneuver a surgical tool and the hand-centric controller via their right hand. A cannula is presented in FIG. 2 as an example of a surgical tool for simplicity in explanation. A suction cannula can be affixed to the hand-centric controller. The cannula may include a suction pump attachment end 202 (e.g., adjacent the outer portion of the hand-centric controller) and a suction tip end 206 (e.g., extending from the hand-centric controller inward). A suction control opening 204 may be positioned along the cannula between the outer and inner portion of the base of the hand-centric controller. The suction cannula may be a suction device removably affixed to the base and intended to remove fluid and tissue from the surgical site as the case proceeds, keeping the site operable and removing pathology. The suction cannula may be used for a very high percentage of the time during a given procedure. The suction cannula can operated by holding it in one hand typically between the thumb and first finger—as shown in FIGS. 2 and 3—such that the suction tip 206 can readily positioned and oriented via comfortable hand movement and the suction control opening is in proximity to the user's thumb.

The distal end of the instrument may be the suction tip 206, directed by the surgeon's hand movements to enter the surgical site where suction is needed as determined by the surgeon viewing the site and surroundings. In some embodiments, the proximal end of the instrument (e.g., suction pump attachment end 202) may connect to a suction tube which is typically connected to a suction source such as a motorized pump. By adjusting the amount of the suction control opening that is covered by the user's thumb, the amount of suction can be varied at the suction tip 206 from near-zero or low suction to the maximum suction possible by the system.

The right hand of the operator may be placed such that the suction pump attachment end 202 of the surgical tool may be oriented to the right of the right hand, and that the thumb of the right hand may be placed close to a suction control opening 204 of the surgical tool (e.g., to allow the operator to easily control suction). Furthermore, the operator may use their index finger to maneuver or guide the suction tip 206 of the surgical tool while also being able to hold and access the hand-centric controller. Although not shown, a sterility drape and/or glove may also be used to provide sterility between the hand-centric controller and the surgical tool.

FIG. 3 is a perspective view of the hand-centric controller for the left hand of FIG. 1 and the hand-centric controller for the right hand of FIG. 2. For example, an operator may utilize, with their left hand, a surgical tool and hand-centric controller designed for their left hand 302, and simultaneously utilize, with their right hand, another surgical tool and hand-centric controller designed for their right hand 304.

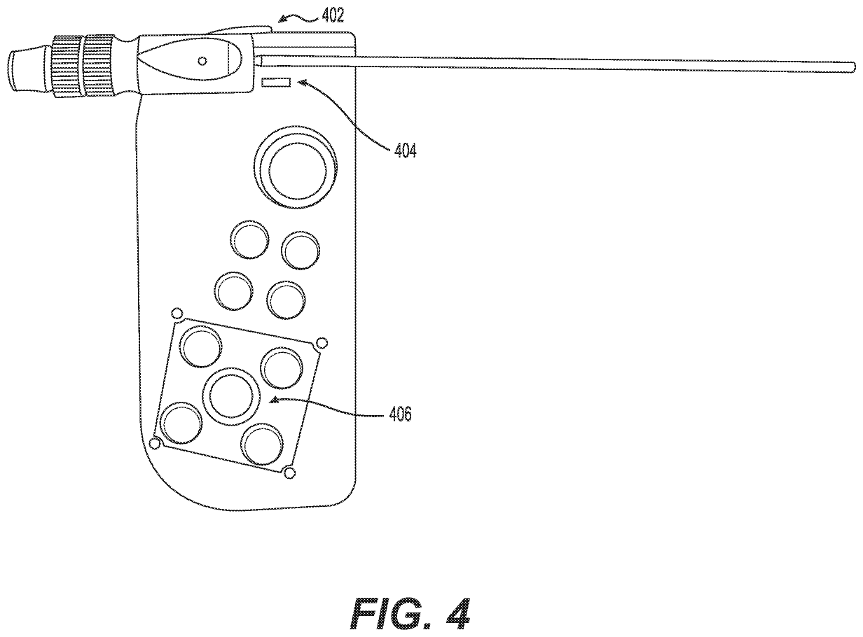

FIG. 4 is a perspective view of an expanded controls hand-centric controller for a left hand, according to another example embodiment. For example, the hand-centric controller may further include an unlock button 402 (also referred to as “unlock joystick” button) to allow accessibility to the joystick. In some aspects, the unlock button may activate other push buttons, as will be discussed herein. The hand-centric controller may include a plate that can be removed to access advanced controls—as shown FIG. 4—that provide additional functions, controls, states, and/or actions. In some embodiments, the advanced controls may include one or more rocker switches 406. For example, the operator may prefer to use rocker switches 406 to maneuver a microscope camera instead of the discrete buttons of the joystick, as the rocker switch may be easier to control. As previously discussed, the hand-centric controller may also include an indicator light 404 to provide feedback from the microscope camera.

In some embodiments, the hand-centric controller with cannula is configured to be used with the user's non-dominate hand to keep the surgical site free from fluids such as blood and excess added irrigation fluid as well as to remove any tissue that has been separated from the patient. The surgeon may use the non-suction-cannula hand to operate the primary instrument for that part of the procedure (e.g., another controller without a cannula, other device, free-hand, etc.).

In current control systems, when the surgeon needs to adjust the microscope, they typically relinquish control of the instruments in one or both of their hands and reach to physically engage with the microscope using those one or both hands. However, this is undesirable as the surgeon's hand controlling the suction cannula is doing a critical job, but does not require the full capability of that hand at all times. The hand-centric controller of the present controller alleviates this issue by providing a surgeon-hand-centric controller for the robotic digital surgical microscope, fitting unobtrusively into the hand while not interfering with normal operation of certain nominally “simple” instruments such as the suction cannula.

The hand-centric controller system enables concurrent control of a surgical hand instrument, such as the suction cannula along with the hand-centric controller for the robotic digital surgical microscope. The hand-centric controller may include a fastening member to secure the controller to an instrument temporarily such that the use of the instrument and the use of the controller are both not impeded and the controller is kept in a secure position and orientation relative to the instrument. In some embodiments, a channel is provided as part of the upper body of the hand-centric controller into which channel fits a portion of various surgical hand instruments such as the shaft of a suction cannula. In some embodiments, a small disposable device is pre-attached to multiple surgical tools, and the controller snaps onto the disposable device. This arrangement allows the controller to be quickly switched between many tools. In this case, the controller may be designed symmetrically so it can be placed on either right or left handed tools.

A resilient conforming material may be installed in the channel and the channel sized appropriately to enable “gripping” instrument parts of various sizes and shapes. The gripping material may be configured to grip even when a surgical drape is installed between the controller and the instrument to maintain sterility of the instrument. In one embodiment, the gripping material and the hand-centric controller are configured to exert pressure by the resilient material upon the instrument part it is fully or partially encapsulating thereby offsetting the reduced friction between the resilient material and the instrument by the (typically thin film plastic and somewhat slippery) sterile drape. In other cases, the controller may be single-use and sterile, so no drape is required.

The hand-centric controller is configurable to accompany/interface with a wide variety of different instruments used in the non-dominant hand during surgery with a robotic digital surgical microscope. In some embodiments, the hand-centric controller is configured to provide the user's hands with access to a robust set of controls for the robotic digital surgical microscope. The hand-centric controller may include a set of control components that provide user interface to, and control of, the robotic digital surgical microscope.

The hand-centric controller may be placed in proximity to and/or within easy reach of the natural position of the user's fingers (including the thumb). Further, the hand-centric controller may be configured to allow the user easy interaction with these control components, thus enabling the user to keep both hands on instruments and keep both hands in the surgical field for a high percentage of the procedure. This speeds the procedure and smooths the workflow.

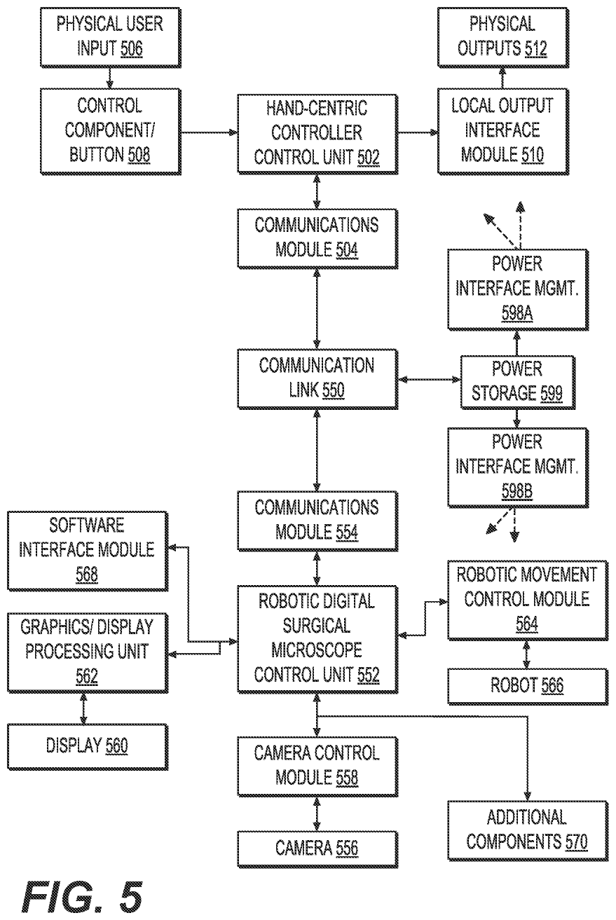

FIG. 5 is a block diagram illustrating a hand-centric controller system, according to an example embodiment. In one embodiment, the hand-centric controller system may include a hand-centric controller unit 502 and robotic digital surgical microscope control unit 552, which may each respectively control, manage, or otherwise process functions of other subcomponents associated with the hand-centric controller and the microscope camera, respectively. The hand-centric controller unit 502 and robotic digital surgical microscope control unit 552 may be communicatively coupled through a communication link 550 (e.g., a cable link, a wired or wireless network, etc.). The hand-centric controller unit 502 and the robotic digital surgical microscope control unit 552 may each include a communications module (e.g., reference labels 504 and 554, respectively) that allows the respective unit to access the communications link 550. Power interface and management units 598A-598B may be used to supply power to the hand-centric controller unit 502 and robotic digital surgical microscope control unit 552, respectively, or their respective subcomponents. In some aspects, the power supply may be stored in a power storage 599 (e.g., a rechargeable battery). The power supply may be monitored and appropriately channeled to the devices by the power interface and management units 598A-598B.

As previously discussed, the hand-centric controller may allow an operator to enter physical user input 506 (e.g., pressing, toggling, moving, switching, etc.) within finger's reach (e.g., while the operator is using a surgical tool or is otherwise engrossed in surgery). The physical user input 506 may be received by a control component 508 (e.g., thumbstick or joystick, push button, toggle button, etc.) (also referred to as a “control element” or “button”). The control components that receives user input and output signals may be referred to as buttons for simplicity and may include both non-discrete buttons (e.g., push buttons, toggle buttons, rocker switches, etc.) and discrete buttons (e.g., motions of a joystick or thumbstick, motions of a head tracking device, etc.). The control component and/or buttons 508 may activate signals that may be received by the hand-centric controller control unit 502. Furthermore, the hand-centric controller control unit 502 may also receive external signals. For example, the hand-centric controller control unit 502 may receive feedback from the robotic digital surgical microscope control unit 552. Such feedback may include, for example, an inability for the microscope camera to execute a requested action, a low battery status, a poor connection, a lack of or loss of connection, etc. The hand-centric controller control unit 502 may deliver this feedback to the local output interface module 510, which may determine whether and how to provide an indication of this feedback via physical outputs 512 (e.g., LEDs, lights, sounds, haptic feedback, vibrations, etc.). In some aspects, the local output interface module 510 and the associated software and hardware used to deliver the physical output of a feedback received by the hand-centric controller control unit 502 may be referred to as a programmable feedback module.

As previously discussed, the microscope camera may allow an operator to visually inspect various detailed aspects of a surgical site, and the hand-centric controller may be used to control the microscope camera seamlessly from the reach of the operator's hands. The camera 556 of the microscope camera may scan or capture image data from a field of view. A camera control module 558 may include one or more processors and/or instructions for controlling the optical, mechanical, and/or lighting of the camera 556. The camera control module 558 may receive higher level instructions from the robotic digital surgical microscope control unit 552 (e.g., based on requests sent by the hand-centric controller control unit 502) and process, filter, and/or translate those instructions to lower level instructions for the camera 556. A display 560 may output a graphical and/or visual presentation of an image and/or video corresponding to the image data captured by the camera 556. A graphics and display processing unit 562 may be used to output the image or video based on the image data provided by the camera 556 as well as recognize, from the image and/or image data, relevant features (e.g., to track or target locations within the surgical site). A robot movement control module 564 (also referred to as an actuated control system) may be a software or hardware subcomponent of the microscope camera responsible for processing requests to cause one or more components of, or the entirety of, the microscope camera to move. The robot 566 may comprise one or more actuator or other electrically powered movable member (e.g., motors, levers, gears, etc.) that causes the movement based on requests sent by the robot movement control module 564. In some aspects the movement may include the movement of the lens of the camera along an X-axis, Y-axis, or Z-axis. The robotic digital surgical microscope control unit 552 may be further associated with a software interface module 568 that may allow the microscope camera to be programmable to perform different functions described herein (e.g., via different control components of the hand-centric controller). It is to be appreciated that the hand-centric controller system need not include all of these components. Furthermore, the hand-centric controller system need not be limited to the components described. For example, additional components 570 used by the robotic digital surgical microscope control unit 552 may include, but are not limited to: an EDSM, a physical input/output interface, long-term memory, a touchscreen interface, a keyboard, a mouse, a trackpad, and/or a footpedal.

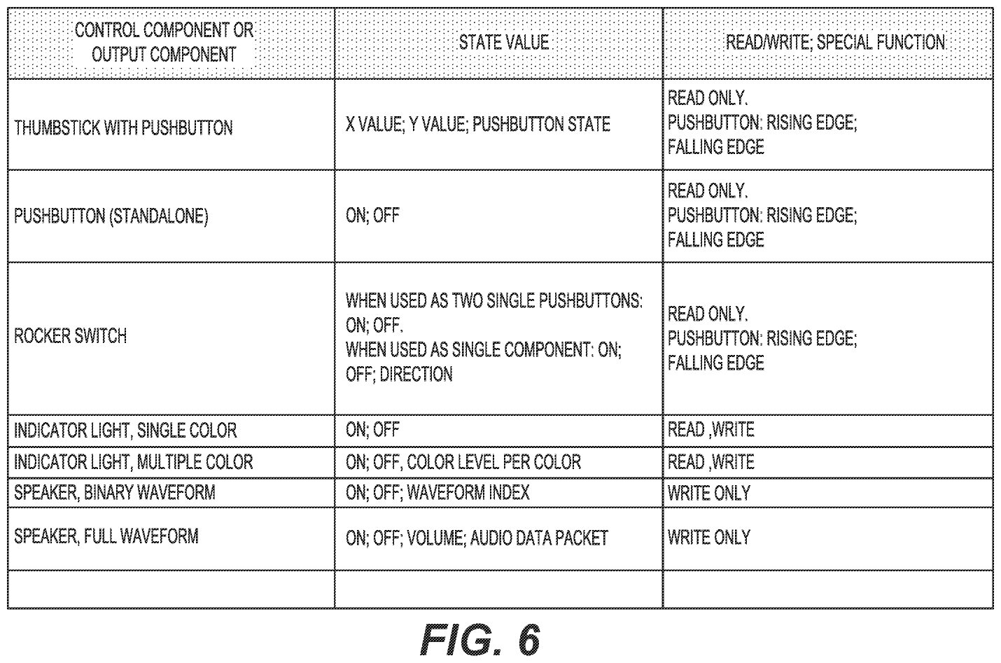

FIG. 6 is a table illustrating control components at various state and values for a hand-centric controller system, according to an example embodiment. As illustrated in FIG. 6, the control components on the hand-centric controller may comprise extensions of existing controls, such as zoom in/out, focus in/out, white light level up/down, and microscope movement, among other controls. In some embodiments, the hand-centric controller includes one or more analog thumbsticks (also called “joysticks”) which enable conversion of the user's two-dimensional thumbstick movement into a two-dimensional vector via the thumbstick hardware and some hardware and firmware on the controller control unit and/or software on the microscope control unit. The thumbsticks/joysticks may provide a pushbutton switch built into the stick itself, actuated by pressing downward along the stick shaft. In some embodiments, the hand-centric controller includes toggle buttons, simple switches and pushbuttons, trackpads, indicator light(s), tactile features enabling navigation about the controller without looking at it, wall(s) or raised boss(es) separating groups of control components meant to control different features, speakers to provide acoustic feedback to the user, and/or vibration motor(s) enabling tactile feedback for operations such as “robot can move no further in the current movement direction.” In some embodiments, the hand-centric controller includes a control unit that is a microcontroller such as the Microchip ATxmega16A4U 8-bit microcontroller with multiple digital inputs/outputs (I/O), analog-to-digital converter (ADC) inputs and universal serial bus (USB) module or the Microchip ATSAME70Q21 32-bit microcontroller with multiple digital inputs, ADC inputs and USB module(s); or Bluetooth Low Energy communication chips.

The analog joysticks/thumbsticks may be configured to use ADC inputs to read their values. Simple switches, toggle switches and pushbuttons may be connected with minimal interface components to the microcontroller's digital inputs. Some indicator lights are driven directly from some of the digital I/O while some others use a buffer amplifier which is driven from digital I/O. Speakers are each driven from a digital-to-analog converter (DAC.) In some embodiments, a digital output can be operated to simulate a DAC for simple sound types, connecting directly to a speaker or through a buffer amplifier. Additional embodiments of control components or output components are illustrated in FIG. 6.

In some embodiments, the hand-centric controller control unit communicates with the control unit of the robotic digital surgical microscope to control the operation of said microscope. In some embodiments, the hand-centric controller control unit is a microcontroller containing one or more USB communications modules. The state of the control buttons is gathered by the microcontroller in a continual polling manner in some cases and on an interrupt basis in other cases. The current state of the control buttons is communicated on a continual basis to by the microcontroller via a communications link (e.g., in this case a USB module) to the control unit of the robotic digital surgical microscope. In other embodiments, the communications link is via wireless protocol such as Bluetooth, and the state of the control buttons is communicated via that link. Communications in the other direction (e.g., from the control unit of the robotic digital surgical microscope to the hand-centric controller) may also be enabled. For example, indicator lights on the hand-centric controller are controlled by the software program(s) operating in the control unit of the robotic digital surgical microscope and/or controlled by the hand-centric controller control unit. In some embodiments, the communications format adheres to the standard used to implement the physical communications link. In one embodiment, the communications format is USB 2.0, which permits half-duplex communication (e.g., bi-directional but only one direction active at a time.)

The hand-centric controller may implement a user interface to existing functions in the robotic digital surgical microscope. Additionally, there may be commands that are specific to the hand-centric controller which are not used by the microscope when the controller is not in use. The commands communicated between the hand-centric controller and the control unit of the microscope are mostly but not wholly generic to the microscope usage; some are specific to the hand-centric controller only.

The hand-centric controller may be powered by battery and/or cabled power supply. The hand-centric controller control unit has the power it needs to operate its electronics and may include back up power components. Power and ground circuitry may be provided to all control components by the power management module of the hand-centric controller. In some embodiments, the hand-centric controller control unit obtains power from USB when USB is the communications link. For example, USB 2.0 provides ample power (including voltage and current) capability to supply the hand-centric controller. In some embodiments, the communications link is wireless such as Bluetooth. In those embodiments, the hand-centric controller contains a battery module which provides power. The battery module contains one or more rechargeable batteries and provides a port to connect a charging unit for recharging the battery or batteries at regular intervals determined by usage and normal non-use-related battery drain. In one embodiment, the battery or batteries are single use and are replaceable via an operable door which enables access to the battery compartment. In some embodiments the entire unit, including the battery, is single use and disposable.

In some embodiments, the hand-centric controller is configured to conform to sterility standards for the operating room. In some embodiments, the hand-centric controller is draped with a drape of appropriate size and shape before use and remains draped for the entirety of the procedure. The drape may be thin and transparent such that it does not interfere with the operation of the controller in any way. To account for the typically slightly slippery nature of the drape, the drape may be secured in two places near the body of the controller. Additionally, the securing device may be configured to exert more pressure on the part of the instrument. In some embodiments, the hand-centric controller may be made to be single-use and may be sterilized after manufacture, e.g., before packaging. In those embodiments, no drape may be needed and the design of the instrument grip can be adjusted slightly to allow for an amount of pressure on the instrument appropriate to enable easy instrument insertion into and removed from the grip while also enabling secure holding of the instrument during controller use.

In an example method of use for the hand-centric controller, a patient's surgical site, operating room surroundings and attending surgeon(s) and assistant(s) are prepared to conform to operating room sterility standards. One such assistant is the “scrub nurse” who is fluent in the knowledge and language regarding surgical instruments and procedures and is the assistant usually working closest to the surgeon physically and usually giving the surgeon the most direct assistance in the form of selecting, delivering to and relinquishing from the surgeon instruments and actions requested by the surgeon.

The method continues while the anesthesia equipment, patient monitoring equipment and associated personnel are prepared and set to use. Hand-operated instruments required for the procedure are sterilized, transported to the now-sterile area surrounding the patient and made available to the scrub nurse for eventual delivery to the surgeon upon request at various times during the procedure.

The method continues with powered instruments being prepared and their critical parts sterilized and brought close to or into the sterile area. The suction cannulas are prepared: one highly-used set of instruments is the set of suction cannulas of various properties such as varying diameter. During the powered instrument setup step, the suction pump and tubing are set up which provide suction and a destination for that which is sucked. The tubing may be connected to the cannula base which cannula is indicated for first use during the procedure. Sometime before first suction is required, the suction pump is turned on.

The robotic digital surgical microscope is prepared in its standard way for use including: (1) the microscope is powered on; (2) basic functionality is verified; (3) the microscope is draped with a sterile drape; (4) the microscope is placed near the surgical site but still far enough away to require “gross microscope movement” before the microscope can view the surgical site within the microscope's field of view. The hand-centric controller is removed from storage if necessary (for example from the storage drawer built into the microscope).

Next, the hand-centric controller is powered and connected informationally to the control unit of the microscope. In some embodiments, the power and communications are provided via USB. In these embodiments, the controller is plugged into a USB port accessible on the external part of the microscope, thereby applying power to the controller and connecting it informationally to the microscope. In some embodiments, the power and communications are via USB. In this embodiment the controller is plugged into a USB port accessible on the external part of the microscope, thereby applying power to the controller and connecting it informationally to the microscope. In some embodiments, the communication connection is wireless between the controller and the microscope and the controller is battery-powered. In these embodiments, a power button is switched on and a “connect” button is pressed on the controller which initiates a connection procedure as well as initializing the controller for use. In some embodiments, these actions are combined into a single button. An optional indicator light may be positioned on the hand-centric controller to indicate that the controller is ready for use.

The hand-centric controller may be draped with a sterile drape. In some embodiments, the controller is shipped as sterile in a package and is unpackaged by sterile personnel before the aforementioned connection step. The first suction cannula to be used during the procedure is inserted into the controller's grip feature. The controller and cannula are now ready for use by the surgeon. Typical procedures requiring the use of the robotic digital surgical microscope contain one or more steps prior to requiring the microscope. During these steps it is preferred to have the microscope near the patient but typically still too far away for the surgical site to be in the field of view of the microscope. Often, the microscope cart needs to be wheeled toward the patient once the microscope is needed. The cart is then optionally locked in place by locking two or more of its (typically four) wheels.

Finally, a set of gross microscope movements typically using just the robotic arm (and not cart movements) is required to move the microscope head to be “in the ballpark” by which it is meant that the field of view of the microscope (as measured by viewing on the microscope display(s)) is close to what it needs to be to perform the next step in the procedure.

It should be noted that in much of the description the surgical tool was specified as the suction cannula. However, the controller can be placed on any surgical tool, including, but not limited to, tweezers, forceps, or electrocautery devices. Also or alternatively, the controller may be built into one of the surgical tools for the same effect. Also or alternatively, the control device need not be attached to a surgical tool, and may be attached to a “ring” that goes around the surgeon's finger or palm, or attach to the surgeons glove. Furthermore, any movement of the surgical controls from the microscope into the sterile field.

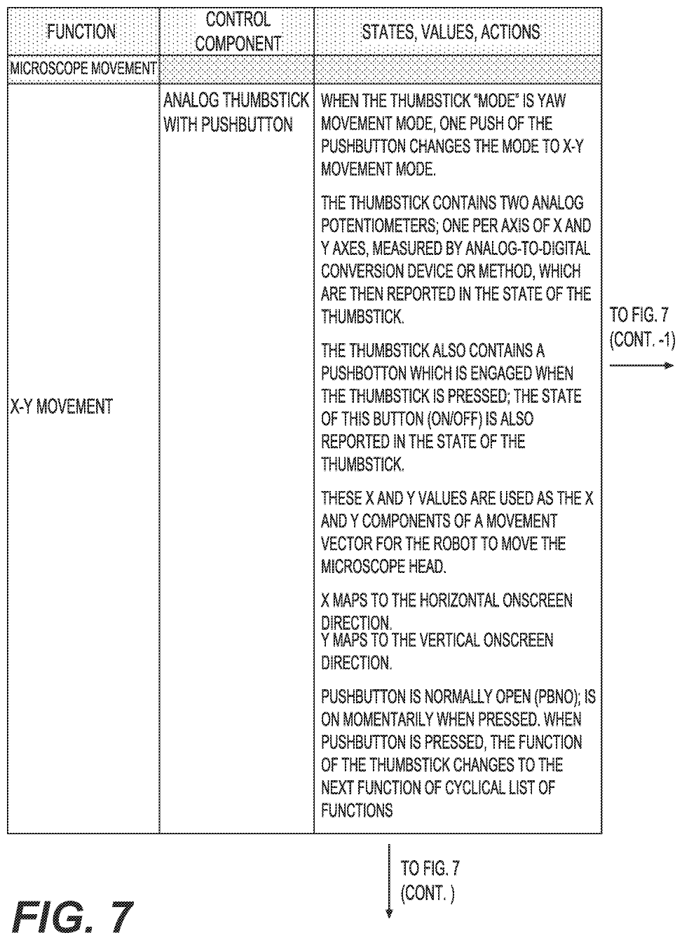

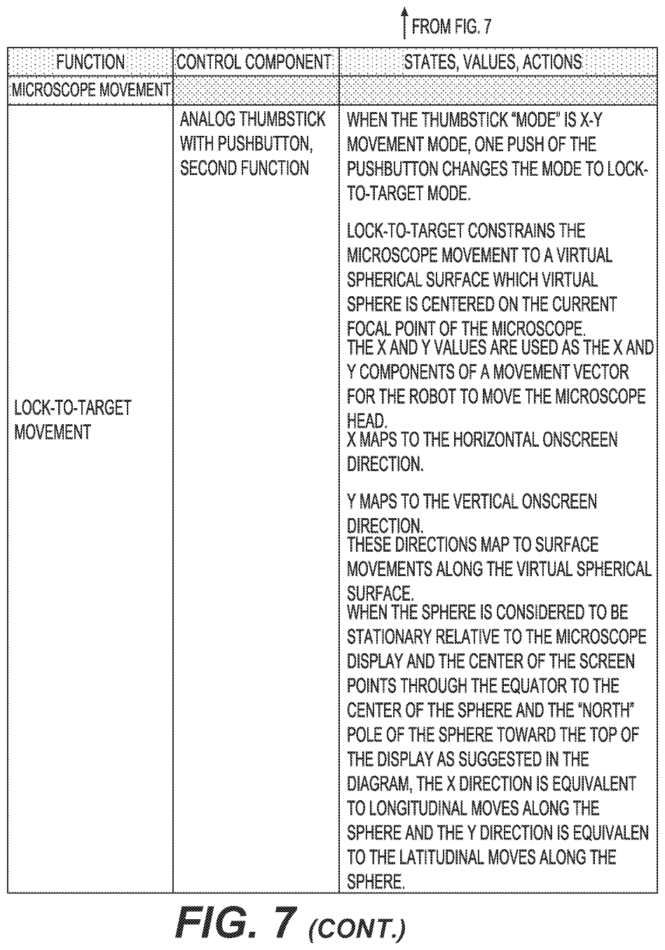

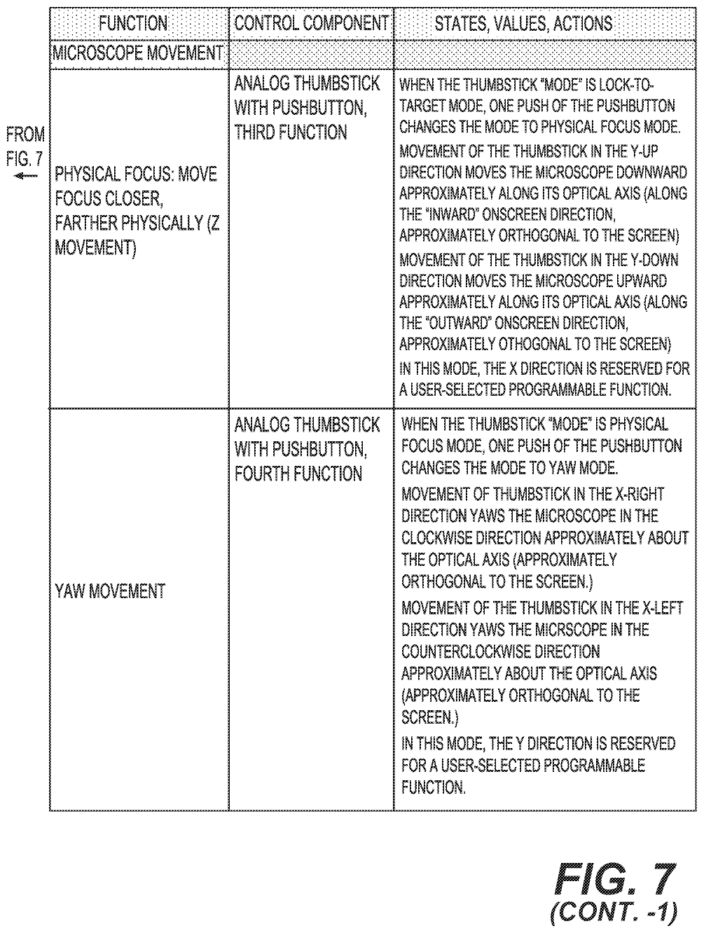

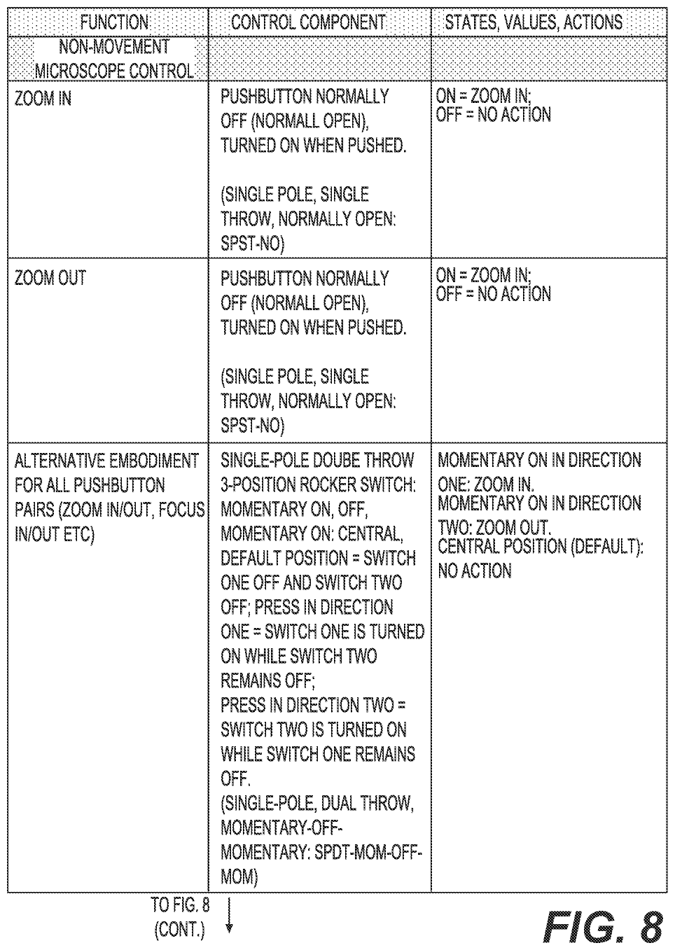

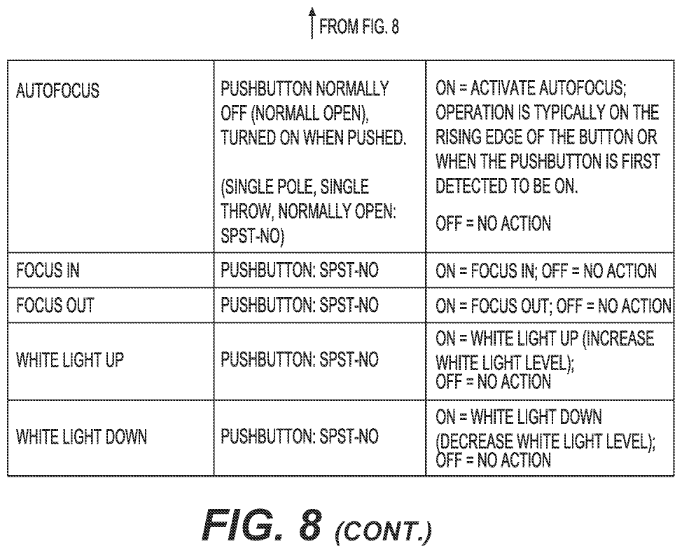

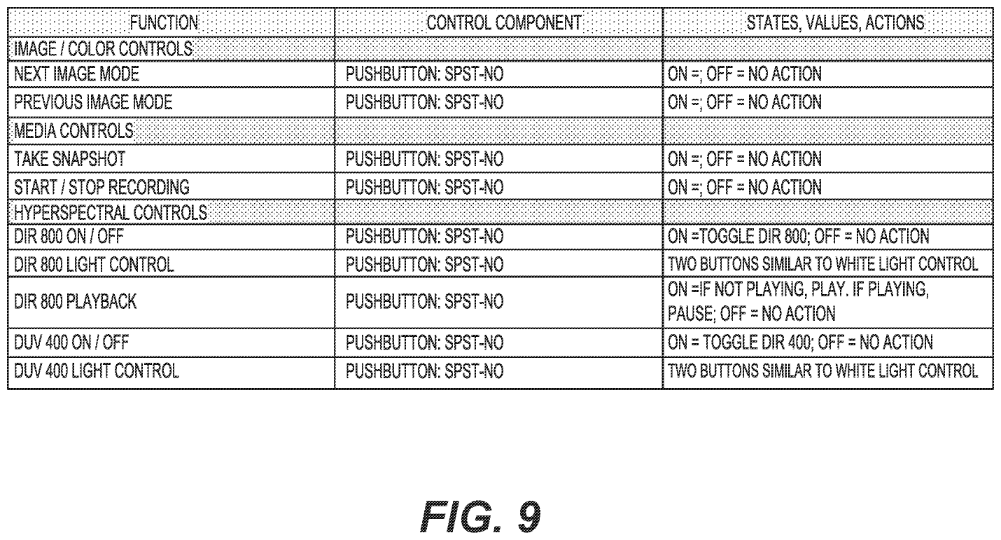

Referring to FIGS. 7-9, a plurality of functions (e.g., microscope movement controls, non-movement microscope controls, image/color controls, media controls, and hyperspectral controls) and corresponding control components, states, values, and actions with respect to how operation of the hand-centric controller for the robotic digital surgical microscope is used to enable the user to control a majority of the microscope functions using a single hand are illustrated.

In some embodiments, before and during use, the hand-centric controller and associated software running on the microscope control unit provide the means to configure the controller for user preference, different instrument accompaniment and procedure appropriateness. Some configuration may be completed prior to use and some may be available during use. In some embodiments, before use, the hand-centric controller may be configured to accept the first surgical instrument that the controller accompanies. The functions of control components may also be specified both for user preference and procedure appropriateness. In some embodiments, there may not be a special configuration required to prepare to accept the first surgical instrument to be accompanied because the default configuration supports many types of instruments.

In some embodiments, a reduced-function hand-centric controller is used to simplify the dexterity required to operate the controller. This allows the surgeon to operate in that hand surgical instruments which require higher surgeon hand dexterity than the suction cannula. An example of such an instrument is forceps. The simplified controller in such an embodiment slips over the surgeon's wrist and rests along the palm such that it is operable by the two or three fingers farthest physically from the thumb. This frees the thumb, index finger and optionally the middle finger for higher dexterity control of surgical instruments.

In some embodiments, the hand-centric controller comprises just one or a small number of simple controls such as one pushbutton normally open (PBNO.) The advantages of this approach are: lower cost of the controller; reduced learning curve for the surgeon; and enabling significant microscope control features.

In some embodiments, the user's head is used to control the movement of the microscope by using a commercially available head tracking device and its peripheral devices.

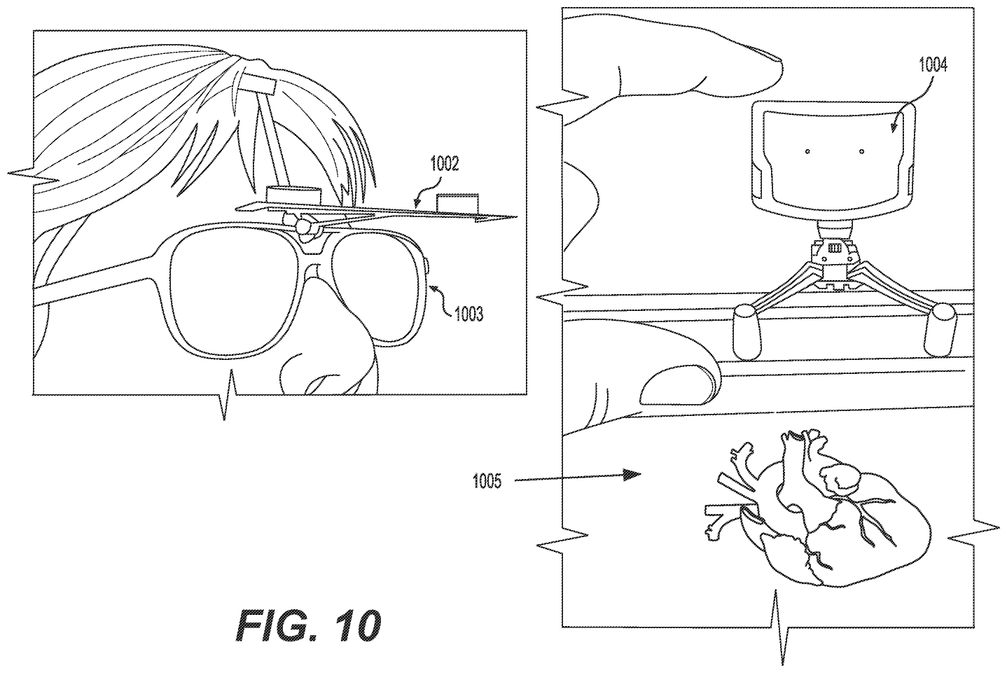

FIG. 10 shows perspective views of a head tracking device mounted on 3D glasses with an integrated camera and light source module of a hand-centric controller system, according to an example embodiment. In some embodiments, controlling the movement of the robotic digital surgical microscope may implement head tracking technology provided by devices such as the TRACKIR 5. The head tracking device may include a reflective target 1002 mounted on 3D glasses 103 required for stereoscopic viewing in some embodiments of the robotic digital surgical microscope. A head tracking device camera 1004 (e.g., TRACK-IR 5 camera) may be mounted on the display 1005 of the robotic digital surgical microscope, facing the user for use with the head tracking device. Since the user may typically be looking at the display 1005 (e.g., while performing surgery), this arrangement provides for improved head tracking over current control systems. In some aspects, the head tracking device camera 1004 may be integrated or associated with a light source module.

The hand-centric controller provides a user with an engage/disengage function of head tracking in the surgeon's hand. The hand-centric controller provides improvements over existing control systems (both for head tracking and/or other control systems) by providing: hand operation of a switch is preferable to foot operation via a footswitch for many surgeons who suffer from too many footswitches in the operating room; hand operation of a simple switch is more reliable with fewer false positives and false negatives than voice control; and removing the reliance on an assistant to perform engage/disengage upon verbal command burdens that assistant with a continual task that lasts through the duration of the procedure.

In some embodiments, the engage/disengage is implemented via a simple hand-centric controller with one pushbutton normally open. The head tracking is engaged when the pushbutton is pressed, and the head tracking is disengaged when the pushbutton is not pressed. An example simple device with two such buttons is the electric pencil normally used for cautery, with the cautery tip removed, and the second button is optionally used to control another function of the microscope. In some embodiments, there are three contacts at the end of the electrical cord; when switch one is engaged, contact one is connected electrically to contact three. When switch two is engaged, contact two is connected electrically to contact three. In some embodiments, the three contacts are connected to a modified hand-centric control unit on which the control components are optionally removed, and which typically resides outside of the sterile field, and not in the surgeon's hand. The switches may be then used as pushbuttons normally open, the function of which type of pushbutton is described elsewhere in this document. In some embodiments, the command payload may be modified to include the status of these pushbuttons to the control unit of the robotic digital surgical microscope, which interprets, dispatches and handles the proper actions to engage or disengage head tracking as the status dictates.

In some embodiments, the head tracking of the hand-centric controller is configured to enable near continuous or continuous normal use of the suction cannula while engaging the head tracking function which enables hands-free robotic microscope movement. The term “hands-free” includes the knowledge that a finger is required to operate the trigger to indicate to the system that head tracking is engaged when the trigger is engaged. In these embodiments, the thumbstick may be enable to be used for functions other than microscope movement. The head tracking device may be able to detect and report changes from a starting “reset” or “zero” position and orientation in six degrees of freedom: X, Y, and Z linear distances and Yaw, Pitch and Roll angular amounts. The reset command can be sent to the software of the head tracking device at any time, thereby updating the “reset” or “zero” position and orientation to be the current position and orientation of the head tracking device target relative to the head tracking device. Subsequent measurements are then reported relative to that reset position. Speed and smoothness of the tracking may be selectable by the user's preference.

In some embodiments, the head tracking device and/or the hand-centric controller are configured to map head moves to microscope moves. In other words, the hand-centric controller system is configured to map the head moves to “screen space” on the display. The surgeon is typically viewing the display head on, facing the display nominally along a vector normal to the display surface, centered roughly on the center of the display surface. Thus, if the surgeon moves their head physically to the right, the scene should move to the left, which is the same as the microscope head moving to its right, as “right”, “left”, “up”, “down”, “in” and “out” are determined by the orientation of the sensors and direction of the optical axis of the digital surgical microscope.

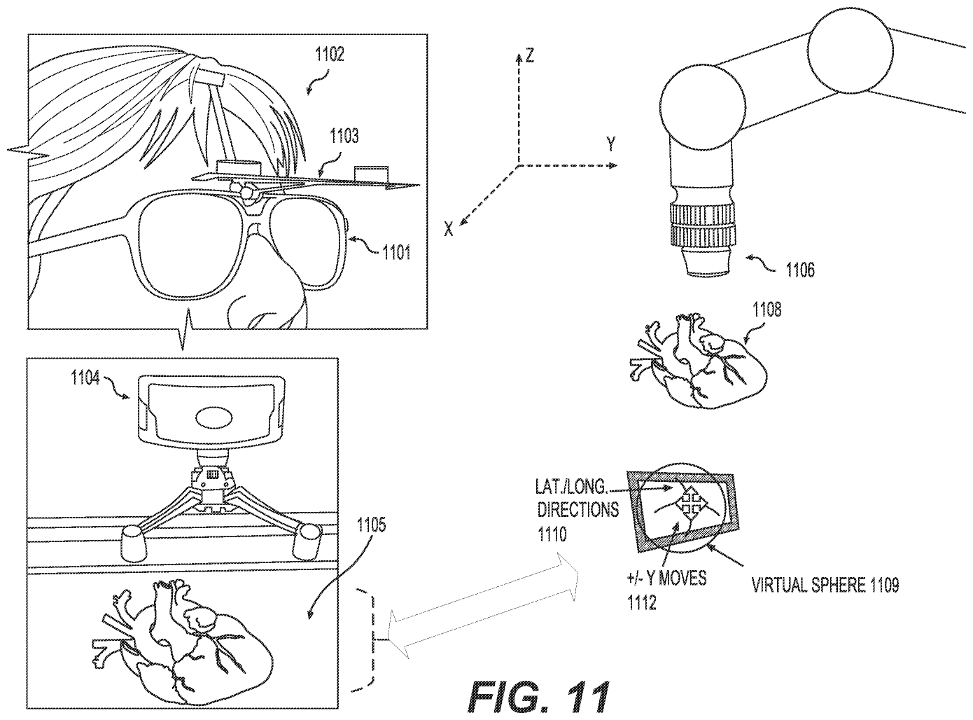

FIG. 11 is a system diagram for head movement mapping of a hand-centric controller system, according to an example embodiment. Referring to FIG. 11, a system for mapping head moves to microscope moves is illustrated. The head tracking device may include 3-D glasses 1101 that are worn on a surgeon's head 1102, and a reflective target 1103 (e.g., TRACK IR 5 target) mounted on the 3-D glasses 1101. The surgeon may view a display 1105 showing a field of view of a surgical site 1108. The display may include a head tracking device camera 1104 (e.g., TRACK-IR 5 camera) mounted on top of it. The display 1105 may be associated with the microscope (e.g., as an output device for image data captured by the microscope camera). In some aspects, the head tracking device camera 1104 may include a light source module. The light source module may be used to detect movement of the head tracking device (e.g., via a reflective target 1103 on the 3-D glasses 1101).

FIG. 11 shows a mapping of head moves along the X-axis or Y-axis to microscope camera moves (e.g., and more specifically the movement of the microscope camera head 1106). As the surgeon's head 1102 moves along the X-axis in a specific direction, the microscope camera head 1106 similarly moves along the X-axis in that specific direction, and the display 1105 correspondingly shows a field of view changing in that specific direction. As the surgeon's head 1102 moves along the Y-axis in a specific direction, the microscope camera head 1106 similarly moves along the Y-axis in that specific direction, and the display 1105 correspondingly shows a field of view transitioning along the Y-axis in that specific direction. The system also shows that the surgeon's head 1102 can move in the Z-axis in either direction (i.e., positive Z (+Z) movement and negative Z (−Z) movement). A positive Z (+Z) move is defined as the surgeon moving closer to the display 1105 along a vector normal to the display surface. The positive Z (+Z) move of the surgeon's head 1102 may cause the microscope camera head 1106 to move physically closer to the surgical site along its optical axis or nearly so. A negative Z (−Z) move of the surgeon's head 1102 may cause the microscope camera head 1106 to move in the opposite direction respectively.

Specifically, the display 1105 may show the field of view of the microscope camera as corresponding to an area on a virtual sphere 1109. The surgeon rolling their head to the surgeon's right can map to a lock-to-target longitudinal move to the right (the “east”) along the equator of the virtual sphere 1109 to which lock-to-target constrains microscope head movement (e.g., as shown by latitudinal/longitudinal directions 1110). A roll of the surgeon's head in the opposite direction can map to a lock-to-target longitudinal move to the left (the “west”) along the equator (e.g., as shown by latitudinal/longitudinal directions 1110). In some aspects, rolling of the surgeon's head in the up or down directions can similarly map to a lock-to-target latitudinal moves up (the “north”) and down (the “south”), (e.g., as shown by latitudinal/longitudinal directions 1110). The surgeon pitching and yawing their head map optionally to similar lock-to-target moves of the microscope head. Another option is to reduce, remove, or re-map those movements. One option is to remap small pitch moves to up/down (+/−Y) moves 1112, as such pitches are more natural for the user to make, and +/−Y moves are more common. Larger pitch moves may be mapped to a similar lock-to-target move, with a thresholding and rate-based heuristic used to determine user intent.

As previously discussed, various embodiments of the present disclosure describe a hand-centric digital surgical microscope system, where a surgical microscope camera is controlled via a hand-centric controller. Various hand-centric controllers described in the present disclosure include fastening members that situate the hand-centric controllers on the surgical tool and/or the hand of operator (e.g., surgeon). Furthermore, the various hand-centric controllers may additionally or alternatively have designs and/or interfaces of buttons that enhance accessibility. For example, some hand-centric controllers have buttons on the side of the hand-centric controller that allows easy access by the non-dominant hand of the operator (e.g., to allow the dominant hand to focus on using the surgical tool). Thus, the various hand-centric controllers described in the present disclosure overcome problems associated with traditional control systems by alleviating the requirement by an operator (e.g., surgeon) to take time, focus, hands, and/or concentration away from surgery to operate the traditional control systems.

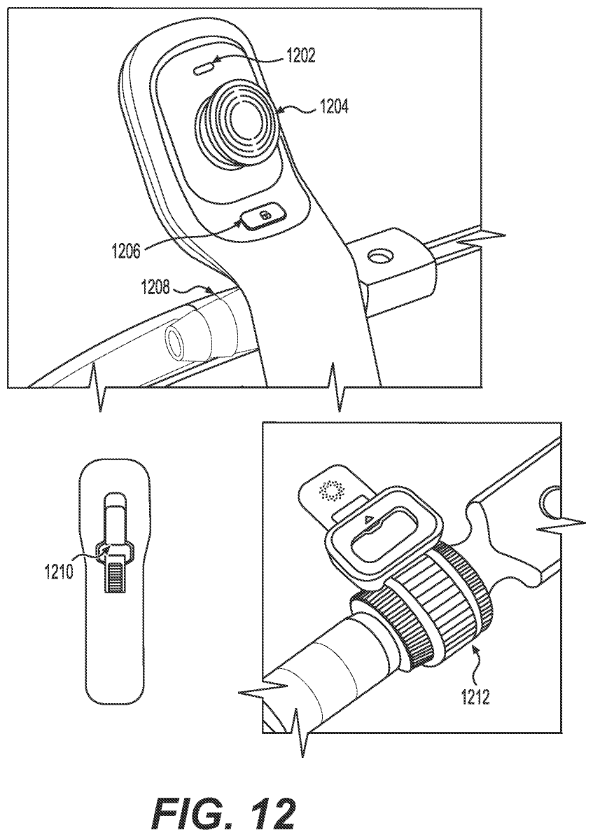

FIG. 12 shows perspective views of a hand-centric controller with a zip tie fastening member, according to an example embodiment. As shown in FIG. 12, the hand-centric controller may include an indicator light 1202 (e.g., an LED) that may function as part of a programmable feedback module of the hand-centric controller. For example, the indicator light 1202 may have constant illumination in a specific color (e.g., in green color) to indicate that there is an electrical connection between the microscope camera and the hand-centric controller. Also or alternatively, the indicator light 1202 may switch to constant illumination in a different color (e.g., red color) to indicate a lack of or loss of electrical connection between the microscope camera and the hand-centric color. Also or alternatively, the indicator light may emit a periodic or flashing illumination, e.g., to indicate another feedback from the microscope camera (e.g., weak connectivity, low battery, device malfunction, etc.). In some embodiments, more than one indicator light may be present on the hand-centric controller to express a variety of feedback from the microscope camera.

Still referring to FIG. 12, the hand-centric controller may further include a joystick 1204. The joystick 1204 may be positioned for the operator to easily maneuver while using the surgical tool, as shown. The joystick 1204 may comprise a plurality of discrete buttons that can be activated by moving the joystick in various directions. Each discrete button may correspond to (e.g., be activated by) a direction of a motion of the joystick. Activation of at least one of the plurality of discrete buttons may cause, via digital communication, a movement of the microscope camera in a direction corresponding to the direction of the motion of the joystick. For example, moving the joystick up may toggle a discrete button for a microscope camera to move in a +y direction across the surgical site, moving the joystick down may toggle a discrete button for the microscope camera to move in a −y direction across the surgical site, moving the joystick left may toggle a discrete button for the microscope camera to move in a −x direction across the surgical site, and moving the joystick right may toggle a discrete button for the microscope camera to move in a +x direction across the surgical site. The movement of the camera may shift the field of view of the microscope camera to a different area of the surgical site. Furthermore, the discrete buttons need not be limited to movement of the joystick in cardinal directions, and the functions of the microscope camera triggered by the discrete buttons of the joystick need not be limited to movement of the microscope camera along the cardinal directions. For example, as previously discussed, functions of the microscope camera that can be triggered by discrete buttons on the joystick may include, e.g., movement along a z axis, a yaw movement, a zoom function, a focus function, the use of lighting, a recording function, a lock to target function, or an image capture function.

Still referring to FIG. 12, the hand-centric controller may further include a joystick lock and/or unlock button 1206. The joystick lock and/or unlock button 1206 may provide a safety mechanism to allow the operator to more freely use the surgical tool by preventing inadvertent joystick movements from triggering unwanted functions on the microscope camera. For example, activation of the joystick lock button (or deactivation of the joystick unlock button) can restrict movement of the microscope camera. Activation of the joystick unlock button (or deactivation of the joystick lock button) can allow use of the discrete buttons of the joystick to cause movement of the microscope camera. In some aspects, as shown in FIG. 12, the lock and/or unlock functionalities may be provided through a single button 1206 that can be toggled from a joystick unlock mode to a joystick lock mode, and vice versa.

Furthermore, the hand-centric controller may be attached to, or attachable to, a surgical tool 1208. For example, the hand-centric controller shown in FIG. 12 may be attachable to the surgical tool 1208 via a zip tie 1212. The zip tie 1212 may be of a significant length to allow it to be secured into a groove 1210 of the hand-centric controller.

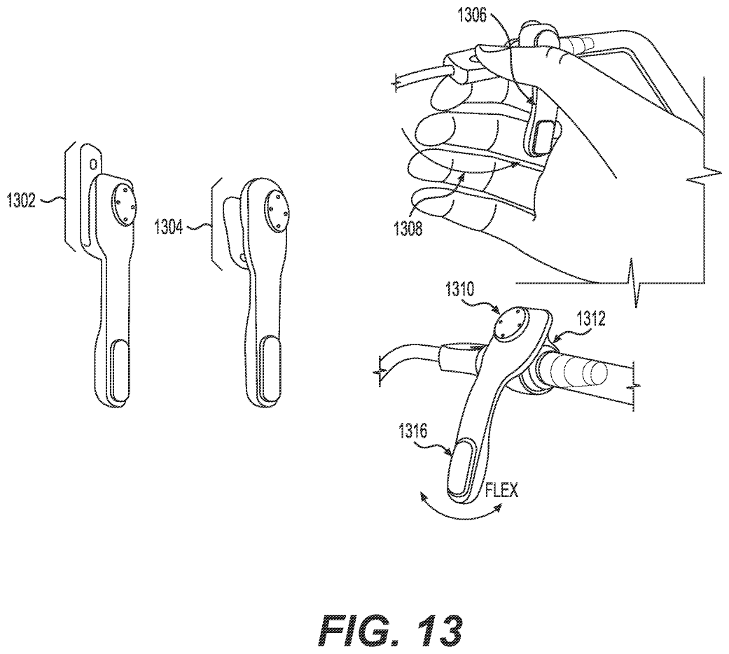

FIG. 13 shows perspective views of hand-centric controllers with a flex connector, according to example embodiments. The hand-centric controllers may include a variety of fastening members (e.g., for attaching the hand-centric controllers to surgical tools), including an adjustable strap 1302 and a clip 1304. The adjustable strap 1302 and/or the clip 1304 may be secured across the back of the surgical tool as shown in reference label 1312. The hand-centric controller may further include a flex connector 1306 and a safety trigger switch 1316. The flex connector 1306 may comprise an elastomeric body housing a flex PCB connecting the safety trigger switch 1316 and a joystick 1310. The joystick 1310 may perform one or more functions as described in relation to joystick 1204 shown in FIG. 12. To use the hand-centric controller (e.g., via joystick 1310), the operator may engage the safety trigger switch 1316, e.g., by using the middle or ring finger (e.g., as shown in reference label 1308).

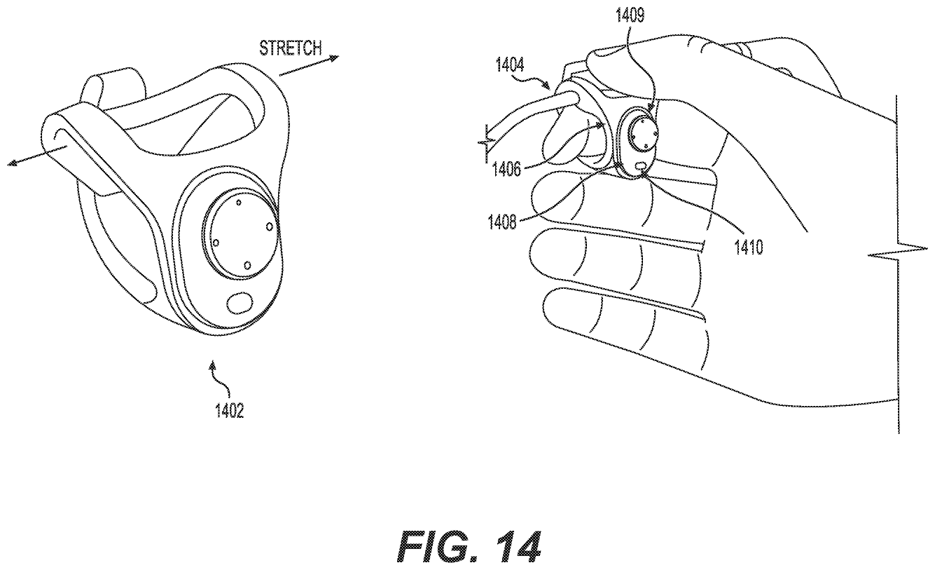

FIG. 14 shows perspective views of a hand-centric controller with an expandable channel, according to an example embodiment. As shown in reference label 1402, the material surrounding the channel of the hand-centric controller may be made of a material (e.g., silicone body 1406) that can be stretched or bent to allow expansion of the channel. As shown in reference label 1404, the hand-centric controller can allow a finger of the operator (e.g., the index or middle finger) to be strapped to a surgical tool (e.g., cannula) with an elastomeric strap. Thus, the surgical tool may be adjacent to and controllable by the finger, and the elasticity of the material can allow movement of the cannula. While the silicone body 1406 may provide elasticity for the channel expansion and can adapt to the finger of the operator to provide a natural feeling, a hard plastic housing 1408 near the joystick 1409 can provide a stable user interface and electronics housing. The joystick 1409 may perform one or more functions as described in relation to joystick 1204 shown in FIG. 12. The hand-centric controller may further include a timer switch 1410. The timer switch 1410 may be used to unlock the joystick 1409, and then automatically lock the joystick after a predetermined duration of time (e.g., a few seconds). The unlocking and locking functions for the joystick may be similar to those described in relation to the lock and/or unlock button 1206 shown in FIG. 12.

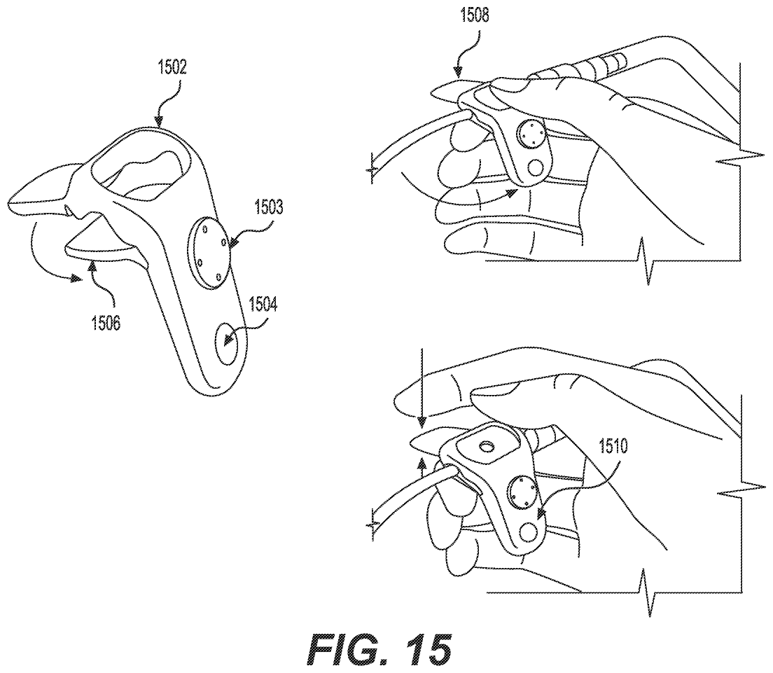

FIG. 15 shows perspective views of a hand-centric controller with a front flap and a rear flap, according to an example embodiment. The front flap and the rear flap may serve as two rigid flaps for different grip situations. The angles of the flaps may be designed to provide freedom of movement and grip, based on different surgical tools. For example, the hand-centric controller may comprise silicon top arms 1502 that may be adaptable to different sizes of surgical tools (e.g., cannulas), and an access door 1506, which may be made of mild durometer material that can open for an operator to insert a surgical tool (e.g., cannula suction hole). The hand-centric controller may include a joystick 1503 performing functions substantively similar to those described in relation to joystick 1204 shown in FIG. 12. A timer switch 1504 (e.g., similar to timer switch 1410 shown in FIG. 14) may be used to unlock the joystick 1503, and then automatically lock the joystick 1503 after a few seconds. As shown in FIG. 15, the operator may secure the controller and the surgical tool by using a rear flap 1508 and a front flap 1510 of the hand-centric controller. For example, the operator may be able to secure the controller and the surgical tool using their index and middle finger to grip the front flap 1510. Also or alternatively, the operator may be able to secure the controller and the surgical tool by squeezing the rear flap 1508 between the index and the middle finger.

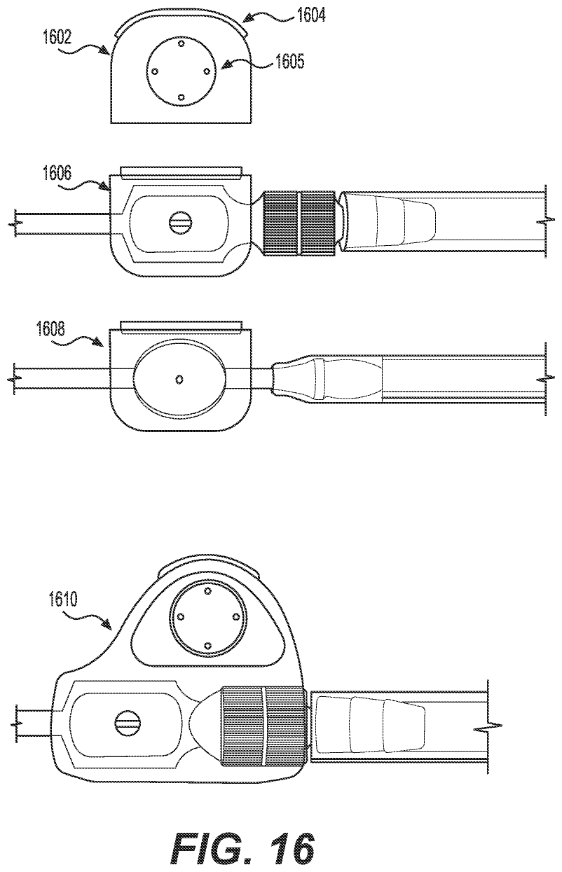

FIG. 16 shows perspective views of hand-centric controllers connected to sleeves, according to example embodiment. The sleeves may allow the operator to insert surgical tools as shown. As shown in reference label 1602, the hand-centric controller may sit directly above the suction hole formed by the sleeve, and may be symmetrical to be suitable for both left and right hands. The hand-centric controller may include a joystick 1605 and a joystick lock and/or unlock button 1604, serving substantively similar functions as joystick 1204 and joystick lock and/or unlock button 1206 of FIG. 12, respectively. The sleeves may be adaptable to fit over different surgical tools. For example, sleeve 1606 may be made of silicone that is molded to fit cannula type 1, and sleeve 1608 may be made of silicone molded to fit over cannula type 2. As shown by reference label 1610, the ergonomic shape of the sleeve may put the joystick in a natural position for the thumb of the operator, and may allow the joystick lock and/or unlock button 1604 to nest comfortably in the curve of the index finger of the operator.

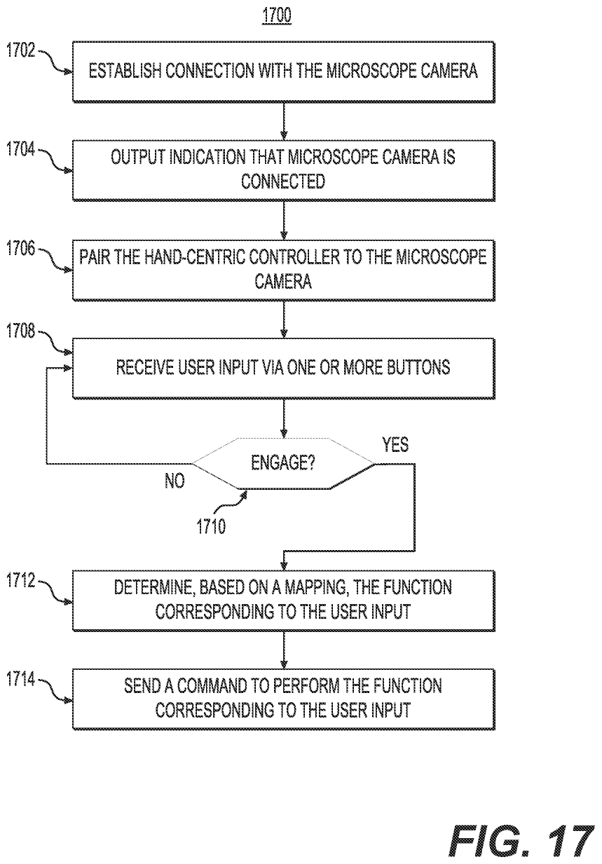

FIG. 17 is a process flow diagram illustrating an example method 1700 of communication between the hand-centric controller and the microscope camera. The hand-centric controller may include one or more processors and memory. The one or more processors may comprise any one or more types of digital circuit configured to perform operations on a data stream, including functions described in the present disclosure. The memory may comprise any type of long term, short term, volatile, nonvolatile, or other memory and is not to be limited to any particular type of memory or number of memories, or type of media upon which memory is stored. The memory may store instructions that, when executed by the one or more processors, can cause the hand-centric controller to perform one or more methods discussed herein, including the example method shown in FIG. 17. The hand-centric controller may include or be associated with one or more components or functions, as described in relation to hand-centric controller control unit 502 of FIG. 5.

Method 1700 may begin by establishing a connection with the microscope camera (block 1702). The hand-centric controller may be located remotely from the microscope camera. For example, the hand-centric controller may be attached to a surgical tool or may otherwise be within reach of the operator. However, the microscope camera may be situated in a position that provides, within its field of view, a display of the surgical site. In some aspects, establishing a connection may be performed by manually forming a wired connection between the microscope camera and the hand-centric controller (e.g., via a USB cable). In other aspects, the connection may be established wirelessly via a wireless network (e.g., via a Bluetooth connection). Examples of wireless networks may include, but are not limited to, a wireless personal area network (WPAN) (e.g., IrDA, Wireless USB, BLUETOOTH, ZIGBEE, etc.) or a wireless local area network. In at least one example, the hand-centric controller may identify, within its network, a device identification of the microscope camera, send a signal confirming a connection, and receive an acknowledgement.

The hand-centric controller may then output an indication that the microscope camera is connected (block 1704). For example, the hand-centric controller may display, via an indicator light and based on an acknowledgment of the connection, an indication that the microscope camera is connected. In some aspects, the hand-centric controller may comprise a programmable feedback module, which may comprise a software and/or hardware component of the hand-centric controller that obtains and outputs relevant feedback from the microscope camera. The feedback may include, for example, an indication that the microscope camera is connection, the microscope camera is not connected, there is poor connectivity, there is low battery and/or power supply, or a user input command cannot be performed (e.g., a user input of moving a microscope camera to a restricted area). The use of an indicator light (e.g., an illuminated or flashing LED on the hand-centric controller) may be one example of an output mechanism that the programmable feedback module may use to indicate feedback. Other examples of feedback may include, but are not limited to, a vibration, an acoustic feedback, a tactile feedback, or a haptic feedback.

At block 1706, the hand-centric controller may pair the hand-centric controller to the microscope camera. For example, the hand-centric controller may include as a software and/or hardware component, a pairing module that pairs the controller to the microscope camera. The pairing may prevent the microscope camera from being controlled by another controller during a time period associated with the pairing, and the pairing may prevent the controller from controlling another microscope camera during the time period associated with the pairing. The pairing module may perform the pairing by storing the device identifier of the microscope camera once a connection has been established, and may cause the microscope camera to deny any requests for being controlled by any other controller during the time period associated with the pairing. In some aspects, the time period associated with the pairing may begin after the connection has been established (e.g., at block 1702), and may remain until the connection ends.

At block 1708, the hand-centric controller may receive user input via one or more buttons associated with the hand-centric controller. The one or more buttons may include non-discrete buttons (e.g., the joystick lock and/or unlock button, the timer switch, etc.), as well as discrete buttons (e.g., movements of the joystick or head tracking device).

For certain user input that is received (e.g., movements of joystick or head tracking device), the hand-centric controller may determine whether it has the permission to engage (block 1710). For example, if a user has previously opted to lock the joystick (e.g., by activating the joystick lock button), the hand-centric controller may not have the permission to engage a user input caused by the joystick. The hand-centric controller may continue to wait until the permission is granted, e.g., via user input.

If, however, the hand-centric controller has permission to engage, the hand-centric controller may determine a function of the microscope camera corresponding to the user input (block 1712). For example, the hand-centric controller may rely on a stored map that links possible user inputs (e.g., activations and deactivations of buttons) to microscope camera functions. The functions of the microscope camera may include, but are not limited to: a movement along an x, y, or z axis; a lock to target movement; a yaw movement; an increase or a decrease in lighting associated with the microscope camera; a zoom function; a focus function; a use of fluorescence; a use of color lighting; a recording function; a hyperspectral function; an image capture function; or a selection of an image mode.

After determining the function corresponding to the user input, the hand-centric controller may send a command to perform the function corresponding to the user input (block 1714). The microscope camera, upon receiving the command, may execute the function. In some aspects, where a function cannot be completed, the microscope camera may send, to the hand-centric controller, feedback on why the function cannot be completed. For example, if a user has previously selected that the field of view of a microscope camera remain within a predetermined area of a surgical site, and the user subsequently inputs (e.g., via a motion on the joystick) a command to move the microscope camera to show a field of view that is beyond the predetermined area, the microscope camera may return a feedback to the controller that the command cannot be completed. In one aspect, the programmable feedback module may indicate this feedback through one or more of a vibration, a tactile feedback, or a haptic feedback.

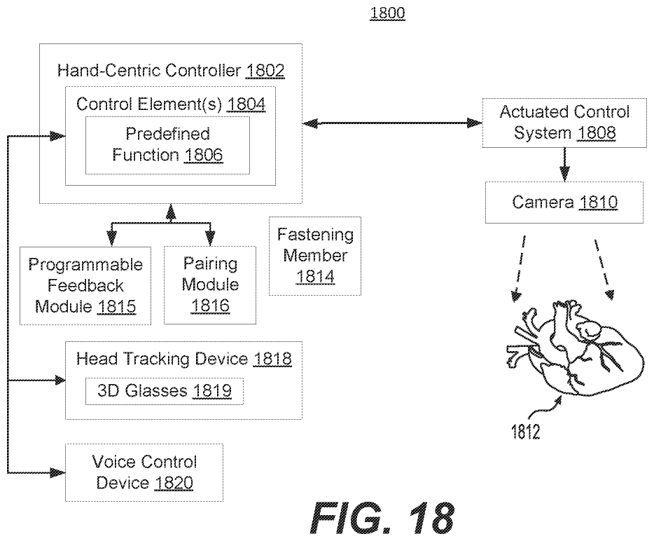

FIG. 18 is a block diagram illustrating a system 1800 for a hand-centric controller for visualization, according to an example embodiment. The system may include a camera 1810 showing a surgical site 1812; an actuated control system 1808 controlling the camera's field of view; and a hand-centric controller 1802 comprising at least one control element 1804. The hand-centric controller 1802 may provide a user (e.g., a surgeon) control of the visualization and actuation systems (e.g., the actuated control system 1808 and the camera 1810) via the at least one control element 1804. The actuated control system 1808 and the camera 1810 may be associated with a microscope (e.g., a robotic digital surgical microscope as disclosed herein). Activation of the at least one control element 1804 can cause, via communication, execution of a predefined function 1806. In some aspects, the at least one control element 1804 may comprise one or more forms, as discussed herein (e.g., a joystick, a thumbstick, a directional button, or a track pad). In some aspects, a joystick may include control elements such as a thumbstick, directional button, or a track pad. The at least one control element may be arranged symmetrically so the hand-centric controller 802 can be used identically by either hand. The hand-centric controller 1802 may be located remotely from the camera 1810. Furthermore, the hand-centric controller 1802 may be within a sterile field or may be placed in a sterile field via a draping.

The fastening member 1814 (also referred to as fastener) may be used to attach the hand-centric controller 1802, or render the hand-centric controller 1802 attachable, for example, to a surgical tool, a hand of the user, a railing of a bed, or a combination thereof. The fastening member 1814 may comprise a variety of forms as previously discussed (e.g., zip tie, front flap and rear flap, sleeve, clip, adjustable ring, elastomeric strap, etc.) and may utilize a variety of methods for attaching the hand-centric controller 1802. In some aspects, the surgical tool may include any tubing associated with the surgical tool. In other aspects, the surgical tool may be independent from any associated tubing.

In some aspects, the system 1800 may further comprise a programmable feedback module 1815. The programmable feedback module causes, via the hand-centric controller 1802, and in response to feedback from the camera, a physical output to notify the user of a condition of the camera 1810, the actuated control system 1808, or the hand-centric controller 1802. The physical output may include, for example, a vibration, an acoustic feedback, a tactile feedback, a haptic feedback, or an indicator light.

In some aspects, the system 1800 may further comprise a pairing module 1816. The pairing module 1816 may pair the hand-centric controller 1802 to the camera 1810. The pairing may cause only one camera to be controlled by only one hand-centric controller (e.g., for a pairing session and/or a predetermined duration of time).

In some aspects, the at least one control element 1804 may engage in communication with a head tracking device 1818. The head tracking device 1818 may detect head movements (e.g., via 3D glasses 1819 worn by a user) and may register user inputs based on the head movements, as previously discussed in relation to FIGS. 10 and 11.

In some aspects, the at least one control element 1804 may engage in communication with a voice control device 1820. For example, the voice control device 1820 may detect human voice and register user inputs based on the human voice (e.g., to allow the user to command movements of the camera 1810 through their voice).

In some aspects, the hand-centric controller 1802 can be disposable. For example, one or more parts of the hand-centric controller (e.g., a covering or casing) may be able to be discarded in order to be replaced with new one or more parts, e.g., to maintain sterility. Also or alternatively, the hand-centric controller 1802 may be reusable (e.g., easily or readily sterilizeable).

It should be understood that no claim element herein is to be construed under the provisions of 35 U.S.C. § 112(f), unless the element is expressly recited using the phrase “means for.”

It will be appreciated that all of the disclosed methods and procedures described herein can be implemented using one or more computer programs or components. These components may be provided as a series of computer instructions on any conventional computer readable medium or machine readable medium, including volatile or non-volatile memory, such as RAM, ROM, flash memory, magnetic or optical disks, optical memory, or other storage media. The instructions may be provided as software or firmware, and/or may be implemented in whole or in part in hardware components such as ASICs, FPGAs, DSPs or any other similar devices. The instructions may be configured to be executed by one or more processors, which when executing the series of computer instructions, performs or facilitates the performance of all or part of the disclosed methods and procedures.

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims. To the extent that any of these aspects are mutually exclusive, it should be understood that such mutual exclusivity shall not limit in any way the combination of such aspects with any other aspect whether or not such aspect is explicitly recited. Any of these aspects may be claimed, without limitation, as a system, method, apparatus, device, medium, etc.

1. A system for a hand-centric controller for visualization, the system comprising:
a camera showing a surgical site;
an actuated control system, wherein the actuated control system controls the camera's field of view;
a hand-centric controller comprising at least one control element,
wherein the hand-centric controller provides a user control of the visualization and actuation systems via the at least one control element,
wherein activation of the at least one control element causes, via communication, execution of a predefined function,
wherein the hand-centric controller is located remotely from the camera, and
wherein the hand-centric controller is within a sterile field.

2. The system of claim 1, further comprising a fastening member, wherein the hand-centric controller is attachable, via the fastening member, to one or more of:
a surgical tool;
a hand of the user; or
a railing of a bed.

3. The system of claim 2, wherein the fastening member comprises a zip tie, wherein the hand-centric controller is attached by tightening the zip tie.

4. The system of claim 3, wherein the fastening member comprises a zip tie base, wherein the hand-centric controller is easily attached and removed from the zip tie base.

5. The system of claim 2, wherein the fastening member comprises a front flap and a rear flap, wherein the user attaches the surgical tool or a tubing of the surgical tool to the hand-centric controller by gripping the front flap and the rear flap around the surgical tool or around the tubing of the surgical tool.

6. The system of claim 2, wherein the fastening member comprises a sleeve, wherein hand-centric controller is attachable to the surgical tool or a tubing of the surgical tool via the sleeve enveloping the surgical tool or the tubing of the surgical tool.

7. The system of claim 2, wherein the fastening member comprises a clip, wherein the hand- centric controller is attachable to the surgical tool or a tubing of the surgical tool via the clip hooking on to the surgical tool or the tubing of the surgical tool

8. The system of claim 2, where the fastening member comprises one or more of:
an adjustable ring to enclose one or more of a finger of the user or a palm of the user, or
an elastomeric strap around the hand of the user.

9. The system of claim 1, wherein the predefined function comprises one or more of:
a movement along any of the six degrees of freedom of x, y, z, roll, pitch, or yaw;
a movement on a hemisphere around the focal point;
an increase or a decrease in lighting associated with the camera;
a zoom function;
a focus function;
a use of fluorescence;
a use of color lighting;
a recording function;
a hyperspectral function;
an image capture function;
an unlock button for actuated motion; or
a selection of an image mode.

10. The system of claim 1, wherein the at least one control element comprises one or more of: a joystick, a thumbstick, a directional button, or a track pad.

11. The system of claim 1, wherein the at least one control element is arranged symmetrically so the hand-centric controller can be used identically by either hand.

12. The system of claim 1, wherein the system further comprises a programmable feedback module, wherein the programmable feedback module causes, via the hand-centric controller and in response to feedback from the camera, one or more of:
a vibration,
an acoustic feedback,
a tactile feedback,
a haptic feedback, or
an indicator light.

13. The system of claim 1, further comprising:
a pairing module, wherein the pairing module pairs the hand-centric controller to the camera, wherein the pairing causes only one camera to be controlled by only one hand-centric controller.

14. The system of claim 1, wherein the at least one control element engages with one or more of the following control devices:
a head tracking device, wherein the head tracking device detects head movements of the user and registers user input based on the head movements, or
a voice control device.

15. The system of claim 14, wherein the head tracking device comprises 3D glasses configured to be worn by the user.

16. The system of claim 1, wherein the at least one control element generates a machine vision analysis of the camera image, and wherein the machine vision analysis tracks a location of a tip of the surgical tool in the camera view.

17. The system of claim 1, wherein the hand-centric controller is disposable.

18. The system of claim 1, wherein the hand-centric controller reusable.

19. The system of claim 1, wherein the hand-centric controller is sterile.

20. The system of claim 1, wherein the hand-centric controller is draped.
